(19) United States
(12) Patent Application Publication
(10) Pub. No.: US 2011/0166614 A1
(43) Pub. Date: Jul. 7, 2011

(54) IMPLANTABLE ANALYTE RF SPECTROSCOPY MEASURING SYSTEM

(76) Inventors: Hans Abrahamson, Stockholm (SE); Anders Björling, Solna (SE); Tomas Snitting, Stockholm (SE); Nils Holmström, Jarfalla (SE); Tom Eriksson, Sandviken (SE)

(21) Appl. No.: 12/809,282

(22) PCT Filed: Dec. 21, 2007
(86) PCT No.: PCT/SE07/01157

§ 371 Date: Jun. 18, 2010

(51) Int. Cl. A61N 1/39; A61B 5/1455; A61N 1/362

(52) U.S. Cl. 607/5; 600/310; 607/9

(57) ABSTRACT

An analyte measuring system includes implantable medical device having a RF signal source arranged for generating a RF signal and a transmitting antenna for transmitting the RF signal into a surrounding tissue in a subject body. The system has a receiving RF antenna for receiving the RF signal from the tissue and a signal processor arranged for generating an estimate of a concentration of an analyte in the tissue based on a spectral analysis of the received RF signal.

### BACKGROUND OF THE INVENTION

1. Field of the Invention

The present invention generally relates to implantable medical devices, and in particular to such devices capable of estimating analyte concentration in a subject body.

2. Description of the Prior Art

There is a great need in measuring different analytes in the animal and human body for diagnostic and medical purposes. Today, such analyte measurements are most often performed on blood samples taken from the subjects and analyzed in a laboratory environment. Although drawing a test sample of blood may be simple, it affects the subject's quality of life as he or she must often visit a healthcare facility for taking the sample. In addition, the equipment needed for analysis may be limited to hospital environments. As a consequence, some subjects must therefore make visits to the hospitals on a regular basis.

Several portable sensors implantable in the subject body or at least worn by the subject have been presented. Such sensors are most often based on the local measurement of an analyte in the immediate vicinity of the sensor or even in a measuring chamber of the sensor, through which blood flows. However, such implantable sensors often have significantly limited operational time. A major reason is the formation of connective tissue around the sensor or the measuring chamber. This connective tissue layer prevents or at least severely limits the transport of the analyte close to the sensor. As a consequence, these sensors therefore often become inoperable or highly unpredictable even after a very short operational period.

United States Patent Application Publication No. 2004/0082841 discloses an implantable apparatus for measuring oxygen saturation of hemoglobin in the blood of a patient. The apparatus comprises a light emitter positioned on the outside surface of the pulmonary artery, the aorta or the vena cava. A light receptor is positioned on the outside surface of the blood vessel diametrically opposite to the light emitter. The resulting oxygen saturation measure is then used an evaluation of the patient's pulmonary function. Repeated evaluations could also be used to signal a cardiac pacemaker to increase or decrease its pacing operation in order to accommodate the variations in oxygen requirements of the body during exercise as compared with rest.

### SUMMARY OF THE INVENTION

The present invention overcomes these and other drawbacks of the prior art arrangements.

It is a general object of the present invention to provide an analyte measuring system using RF signals for analyte concentration monitoring.

It is another object of the invention to provide such a system comprising an implantable medical device housing at least the RF transmitter portion of the RF transmitter—receiving functionality.

Briefly, the present invention involves an analyte measuring system having an implantable medical device, such as a pacemaker, defibrillator or cardioverter. The device contains a RF signal source arranged for generating at least one RF signal covering a selected frequency bandwidth. A transmitting antenna is arranged in the implantable medical device or connected thereto, preferably through an implantable cardiogenic electrical lead. The at least one RF signal is forwarded from the source via a connector to the transmitter antenna for transmission into a surrounding tissue. A receiving RF antenna provided in or connected to the implantable medical device or connected to another implantable device or an external non-implantable device, captures the at least one RF signal from the tissue.

The received RF signal is modulated in the frequency domain and therefore has a frequency response (amplitude and/or phase) different from the transmitted RF signal. A signal processor is provided for performing a spectral analysis of the received RF signal to generate an estimate of a concentration of a given analyte in the tissue.

The signal source can be a frequency tunable signal source that sweeps the frequency of the RF signal to basically obtain multiple RF signals of different frequencies in the selected RE range. The signal processor can then analyze the amplitude and/or phase at the different frequencies of the multiple RF signals to estimate the analyte concentration.

Alternatively, the signal source can generate one or more composite RF signals having different frequency components and thereby having a bandwidth in or covering the selected RF range. A bandpass filtering can then be applied to the received signal to obtain frequencies of interest that are analyzed by the processor to get an analyte concentration estimate.

The present invention is based on the finding that different chemical substances have different resonance and absorbance frequencies, which causes a modulation of the transmitted RF signal to form different tops and dips in an absorbance frequency spectrum. By analyzing the amplitude and/or phase changes at a given frequency or preferably at multiple frequencies, the signal processor can determine absolute or relative analyte concentration estimates.

The RF signal generation and transmission of the present invention is preferably performed at multiple different time instances to obtain a trending in the analyte concentration. This concentration trending is of high diagnostic value for physicians and can be used for detecting the onset, relapse or recovery from medical conditions and/or be used for medication purposes.

In a particular embodiment, the transmission of the RF signal is synchronized to specified phases in intrinsic body activity, such as in a selected phase of a respiration cycle or heart cycle. Furthermore, synchronization of signal transmission can instead or as a complement be performed based on body posture of a subject. This transmission synchronization reduces variability and blurring in the received RF signal, where such signal variations are due to the intrinsic body activity and/or body posture. This reduces background noise in the spectrum analysis to thereby improve the accuracy in concentration estimation.

The invention offers the following advantages:

Acquires measurement of blood and tissue constituents without drawing blood from patients;

Acquires HF metrics based on known clinical measurements;

Can be used for myocardial ischemia detection, including silent ischemia; and

Provides an aid in determining drug dosages.

Other advantages offered by the present invention will be appreciated upon reading of the below description of the embodiments of the invention.

### BRIEF DESCRIPTION OF THE DRAWINGS

FIG. 1 is an overview of a subject equipped with an implantable medical device according to the present invention and an external communications unit adapted for wireless communication with the implantable medical device.

FIG. 2 is a schematic block diagram of an embodiment of an implantable medical device according to the present invention.

FIG. 3 is a schematic block diagram of another embodiment of an implantable medical device according to the present invention.

FIG. 4 is schematic block diagram of an embodiment of an analyte measuring system according to the present invention.

FIG. 5 is schematic block diagram of another embodiment of an analyte measuring system according to the present invention.

FIG. 6A illustrates an implementation embodiment of the antennas used in the analyte measuring system according to FIG. 5.

FIG. 6B illustrates another implementation embodiment of the antennas used in the analyte measuring system according to FIG. 5.

FIG. 7 is schematic block diagram of a further embodiment of an analyte measuring system according to the present invention.

FIG. 8 is schematic block diagram of still another embodiment of an analyte measuring system according to the present invention.

FIG. 9 is schematic block diagram of an embodiment of an analyte measuring system according to the present invention comprising a non-implantable device.

FIG. 10 is a diagram illustrating a RF spectrum detectable according to the present invention.

FIG. 11 is a flow diagram of an analyte measuring method according to an embodiment of the present invention.

### DESCRIPTION OF THE PREFERRED EMBODIMENTS

Throughout the drawings, the same reference characters will be used for corresponding or similar elements.

The present invention relates to measuring of analytes and different chemical substances in a human or animal body using an analyte measuring system that comprises an implantable medical device (IMD). The key concept of the invention is to base the analyte concentration estimation on radio frequency (RF) spectral analysis to thereby detect concentration changes of analytes of interest.

The present invention will be a valuable tool in diagnosis and evaluation of subjects having implantable medical devices and can be used as a complement to or instead of traditionally blood sampling analysis. The invention has the potential of being, in real time or close to real time, able to detect sudden changes in analyte concentration in the subject. If the monitored analyte is closely related to a medical condition, an early detection of a severe medical condition is possible even though the subject is not visiting a healthcare facility. Furthermore, by being implemented into the subject, the system of the invention can be utilized for continuous or regular analyte monitoring over time, without the need for a multitude of hospital visits.

The invention is based on the insight that an RF based analyte monitoring has several advantages over the enzymatic and optical techniques traditionally utilized by implantable sensors. The present invention will, thus, operate effectively over extensive periods of time even if connective tissue is growing around the IMD and its RF antennas. The system of the invention therefore does not need to have uninterrupted connection between the antennas and the tissue, where analyte monitoring is performed, as the prior art solutions must.

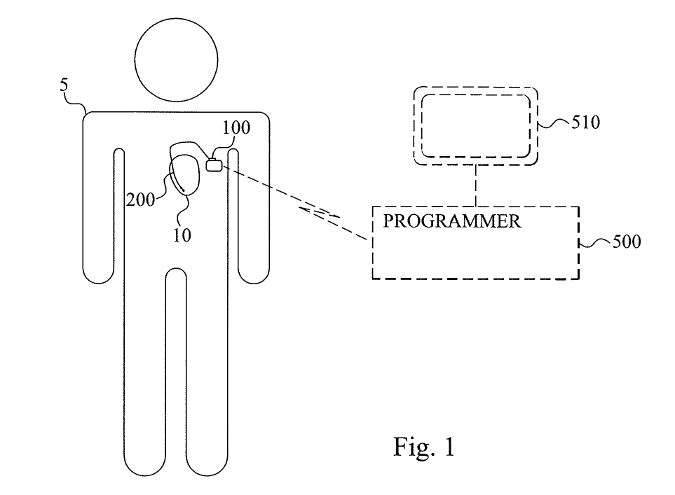

FIG. 1 is a schematic overview of a subject 5 equipped with an IMD 100 connected to the subject's heart 10. The IMD 100 is illustrated as a device that monitors and/or provides therapy to the heart 10 of the patient 5, such as a pacemaker, defibrillator or cardioverter. The IMD 100 is connectable to at least one medical lead 200, such as intracardiac or endocardiac lead, connected to the heart 10.

According to the invention, the IMD 100 has or is connected to at least one RF antenna used for transmitting and/or receiving a RF signal for the purpose of analyte monitoring. As is further described below, the antenna can be provided directly in the IMD case or can or can be arranged on a medical lead 200 connected to the antenna.

In FIG. 1, the IMD 100 has been illustrated as an implantable medical device providing therapy and diagnosis to the heart 10 in the patient 5. However, the present invention is not limited thereto. In clear contrast the analyte measuring system can comprise other forms of implantable devices having access to at least one RF antenna. Examples of such devices include implantable drug pumps, neurological stimulators, physical signal recorders, oxygen sensors, or the like, or even dedicated implantable devices mainly having RF transmitting and/or receiving functionality according to the invention.

FIG. 1 also illustrates an external programmer or clinician's workstation 500 that can communicate with the IMD 100. As is well known in the art, such a programmer 500 can be employed for transmitting IMD programming commands causing a reprogramming of different operation parameters and modes of the IMD 100. Furthermore, the IMD 100 can upload diagnostic data descriptive of different medical parameters or device operation parameters collected by the IMD 100. Such uploaded data may optionally be further processed in the programmer 500 before display to a clinician on a connected display screen 510. In accordance with the present invention, such diagnostic data can include analyte estimations generated by the IMD 100 and/or other raw or partly processed data relating to such analyte estimations. As is further disclosed herein, the programmer 500 or some other external device can constitute one of the units in the analyte measuring system of the invention.

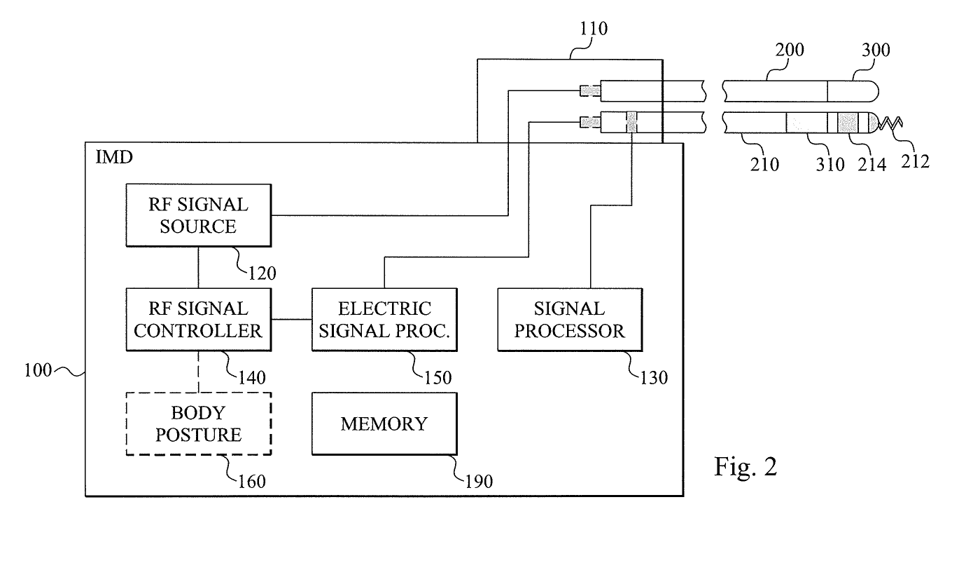

FIG. 2 is a schematic block diagram of an embodiment of an implantable medical device 100 according to the present invention. In this embodiment, the IMD 100 is connected to both a transmitting antenna 300 and a receiving antenna 310. Furthermore, the antennas are provided on medical leads 200, 210 connected to the IMD 100 through a lead/antenna connector 110. The antenna-carrying leads 200, 210 can be provided mainly for housing the RF transmitting 300 and receiving 310 antenna of the invention. Alternatively, at least one of the leads 210 can be an electrical lead connected to the IMD 100 and arranged in connection with a subject's heart for the purpose of providing stimulating pulses, cardioversion pulses, defibrillation shocks and/or sensing electrical signal from the heart. For this purpose, the lead 210 preferably comprises one or more electrodes 212, 214 for electrical signal application and/or sensing. This electrical lead 210 can then be equipped with an RF antenna 310 somewhere along its length.

The IMD 100 includes a RF signal source 120, preferably a frequency tunable RF signal source. This signal source 120 is connected to a transmitting antenna 300 of the device 100. In the illustrated implementation example, this connection is realized through the antenna connector 110 and the medical lead 200 in which the antenna 300 is provided.

The RF signal source 120 is arranged for generating at least one RF signal of a selected frequency range. The frequency range to be covered by the at least one RF signal forwarded to the antenna for transmission to the surrounding tissues is selected based on the particular analyte to measure or monitor. In a preferred embodiment, the range is the in the interval of from about 1 MHz to about 1 THz. This large frequency interval covers spectral changes due to the presence of several analytes that can be of interest for diagnostic and medical purposes.

The following is a short listing of several interesting analytes that can be detected by an IMD 100 or different IMDs 100 according to the invention. The listing should, though, merely be interpreted as containing preferred analyte examples and the present invention is not limited thereto.

### Vitamin K

Vitamin K is involved in the coagulation of blood. More or less all patients suffering from or having been diagnosed for atrial fibrillation (AF) are on some type drug with of anti-coagulatnic effects, e.g. Warfarin or other vitamin K antagonist. These drugs inhibit the synthesis of biologically active forms of the vitamin K-dependent clotting factors: II, VII, IX and X, as well as the regulatory factors protein C, protein S and protein Z. Other proteins not involved in blood clotting, such as osteocalcin, or matrix Gla protein, may also be affected.

Warfarin is very difficult to dose correctly. Patients must usually take blood samples 1-2 times a week to set the correct dosage. The present invention therefore advantageously can be used for monitoring the vitamin K concentration in a subject having an implantable medical device, such as an AF patient. The need for regular blood sampling is then relaxed and the invention can instead provide a regular and periodic monitoring of the vitamin K concentration in the subject body.

### Cholesterol and Blood Fat

Many patients having IMDs such as pacemakers, defibrillators or cardioverters have high blood cholesterol, which is a risk factor for atherosclerosis, principal cause of coronary artery disease (CAD). The IMD of the invention can therefore advantageously be used for monitoring changes in blood cholesterol and blood fats through the RF measurements of the invention.

### Glucose

One of the most common comorbidities of heart failure (HF) patients is diabetes mellitus. For these patients, monitoring the blood glucose value is very important. Currently, the goal for diabetes treatment is to avoid hyper- and hypoglycemia. This is done by lifestyle changes (change of diet, increased exercise etc.) and in some cases insulin. It can be difficult to give right dosage of insulin, and blood tests are needed. The present invention can provide a significant improvement in patient's quality of life by being able to track the glucose level through the RF measurements of the implantable medical device.

The present invention is, by selection of a high frequency RF range, suitable for estimating the concentration of a peptide or protein analyte in the surrounding tissue based on the RF spectrum analysis. Preferred such peptide and protein agents are listed below:

### BNP

B-type (or brain) natriuretic peptide (BNP) has in several studies been shown to correlate extremely well with patients' degree of heart failure. If BNP is measured, this would be a terrific HF metric, already well known by the physician community. By measuring BNP on, e.g. a daily basis the physician would be given unprecedented data of the patient's HF status between check-ups. This would mean quicker follow-up times for the physicians as measurements would already have been made and improved care for the patient as the diagnosis could be made on data not only acquired at the day of the follow-up.

### ANP

A-type natriuretic peptide (ANP) is similar in structure to BNP. It is released mainly in the heart and the release is increased e.g. during atrial distention or stretching. As the left atrial (LA) pressure increases in HF, measuring ANP would lead to the same advantages as BNP. The present invention can therefore be used for ANP measurements.

### Troponin, Myoglobin and Creatine Kinase

When a patient is suspected to suffer from ischemia, blood samples can be taken and analyzed for elevated levels of troponin, myoglobin or creatine kinase. The levels of these are elevated after myocardial damage, e.g. ischemia.

By continually or periodically measuring the presence of these “ischemia markers”, the invention can be used for making a diagnosis faster at the hospital. This also has the potential of detecting so-called silent ischemias (non-symptomatic ischemias). This would improve patient care and episode outcome.

The frequency range utilized by the RF signal source 110 of the invention is preferably in the interval of 10 MHz to 10 GHz. In particular for peptide and protein analytes, the range is preferably selected to be in the interval of from about 100 MHz to about 10 GHz.

It is anticipated by the invention that the RF signal source 120 could be frequency tunable to sweep the frequency of the RF signal over the selected range. This basically results in multiple RF signals having different frequencies the in the desired range. However, the RF signal source 120 can preferably sweep over larger frequency ranges by then transmitting different RF signal of different frequency bandwidths. In such a case, the IMD 100 has the potential of monitoring several different analytes. The RF sweeping functionality of the signal source 120 also has advantageous implementation aspects even if a single analyte is of interest. The reason could be that the analyte has absorbance and/or resonance tops at different frequencies that are not coverable by the limited bandwidth of a single RF signal. The RF signal 120 can then be arranged for providing a signal train of increasing or decreasing frequencies.

In an alternative approach, the RF signal source 120 generates a composite signal consisting of multiple frequencies in the range. Such a more broadbanded signal has a bandwidth that is selected to encompass at least a portion of, possibly the whole RF range of interest.

It is anticipated by the invention that the RF signal source 120 could generate a RF signal of a single selected bandwidth or frequency. This is particular useful if the IMD 100 is mainly utilized for measuring and monitoring a single analyte having an absorbance or resonance frequency in the bandwidth. However, in alternative embodiments, the RF signal source 120 can preferably sweep over larger frequency ranges by then transmitting different RF signals of different frequency bandwidths. In such a case, the IMD 100 has the potential of monitoring several different analytes. The RF sweeping functionality of the signal source 120 also has advantageous implementation aspects even if a single analyte is of interest. The reason could be that the analyte has absorbance and/or resonance tops at different frequencies that are not coverable by the limited bandwidth of a single RF signal. The RF signal 120 can then be arranged for providing a signal train of increasing or decreasing frequencies.

In this embodiment, the IMD 100 has a signal processor 130 arranged for generating an estimate of the concentration of a monitored analyte in the surrounding tissue. The signal processor 130 is connected to a receiving antenna 310, such as arranged on a medical lead 210, through the antenna connector 110. The signal processor 130 performs a spectral analysis of the received RF signal by analyzing the amplitude and/or phase at different frequencies. By detecting changes in the amplitude and/or phase at selected frequencies, the signal processor 130 is able to register changes in analyte concentration and can even be able to calculate absolute concentration values.

In the case of a sweeping frequency signal, the signal processor 130 can utilize all or a portion of the frequencies in the swept range for the purpose of performing the signal analysis. In the case of a composite RF signal with larger bandwidth, the signal processor 130 can have or be connected to a set of one or more filters, such as bandpass filters, for filtering out interesting portions of the received composite RF signal and use these frequency portions in the spectrum analysis.

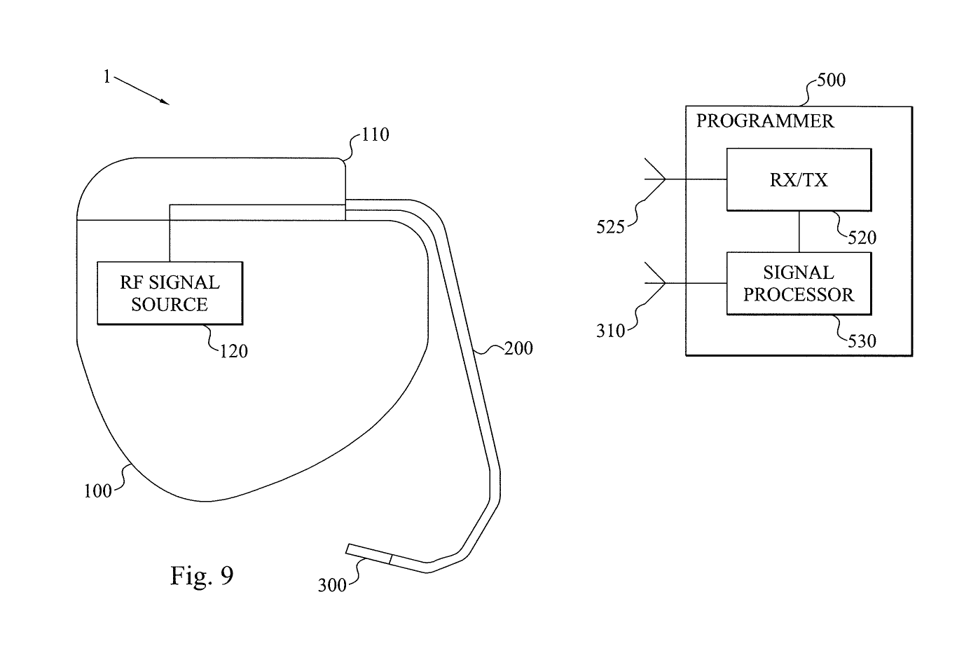

With reference to FIG. 9, the spectral analysis of the processor 130 is preferably performed by investigating the amplitude (phase) of the RF input signal as particular frequencies f₀, f₁ descriptive of absorbance/resonance events due to the particular analyte. The analysis can be performed by measuring the absolute values at these frequencies f₀, f₁. However, such an absolute measurement requires sophisticated calibration procedures in order to map these absolute amplitude/phase values into absolute concentration values. In such a case, the mapping procedure can be performed in a laboratory environment under controlled conditions to get a mapping table or function that allows conversion of measured absolute values into estimates of absolute analyte concentrations. The resulting mapping table or function can then be programmed into the IMD 100 either before implantation or be loaded into the IMD 100 from a programmer (see FIG. 1) after implantation.

Alternatively, the signal processor 130 utilizes a relative absorption/resonance method involving, in addition to the at least one frequency of interest f₀, f₁, a reference frequency f₂. This reference frequency f₂ is selected to have an absorbance that is insensitive to, i.e. does not vary with, the concentration of the analyte. In such a case, the signal processor 130 preferably calculates a ratio of the amplitude at frequency of interest f₀ or f₁ and the reference frequency f₂, such as f_{0,1}/f₂ or f₂/f_{0,1}. The processor 130 then determines an estimate of the analyte concentration based on this ratio.

According to the present invention, estimation of analyte concentration encompasses both an absolute concentration value, such as mol/dm³, g/ml, etc., but also relative concentration representations. Thus, an estimate of analyte concentration also covers a concentration representation specifying an X times increase or decrease in the analyte concentration as compared to a reference value or a previous analyte concentration measurement. For instance, the analyte concentration can be generated based on a comparison of a current spectrum value (amplitude/phase at one or more frequencies or ratio of amplitude/phase at multiple frequencies) and a previously determined spectrum value generated based on a corresponding RF spectrum analysis by the processor 130 at a previous time instance.

The actual frequencies utilized by the RF signal source 120 could be hard-coded before implantation of the IMD 100. However, the present invention also provides for a flexible adaptive RF signal solution. For this reason a RF signal controller 140 is arranged in the IMD 100 connected to the signal source 120. This controller 140 generates, among others, frequency control signals that are employed by the RF signal source 120 for determining the (bandwidth) frequencies of the generated RF signal. This means that the signal source 120 is preferably a frequency tunable signal source 120, where the tunability of the signal frequency is controlled by the RF signal controller 140. The controller 140 can then have access, e.g. in a memory 190, information of desired frequencies for the signal. In a preferred embodiment, this information can be updated through downloading new frequency information data from a non-implantable device, such as the programmer, into the IMD 100. The frequency tunability can therefore be updated even after implantation and during operation of the IMD 100 in a subject body.

The IMD 100 preferably has a memory 190 for storing the determined concentration estimate or representation for later use. The device 100 can then perform new analyte measurements intermittently or periodically and store the different measurement results in the memory 190 for trending purposes. Thus, the IMD 100 will detect significant changes in the analyte concentration by comparing analyte estimates generated at different time instances to thereby be able to, for instance, diagnose a medical detection associated with an increase or decrease in the analyte concentration. The IMD 100 may also utilize multiple generated concentration estimates for the purpose of calculating an average analyte concentration in the tissue. This average concentration is used for diagnostic purposes by comparing a later determined analyte concentration with the average concentration. If there is a significant difference between the concentration estimates, the IMD 100 can store or tag information thereof in the memory 190 for later use. Furthermore, if there is no significance difference between the estimates, the average concentration estimate can be updated based on the newly generated analyte concentration to thereby provide an adaptation in the estimate averaging.

Instead of storing the measurement results in the memory 190 or as a complement thereto, measurement results may be uploaded from the IMD 100 to an external non-implanted device, such as the programmer in FIG. 1. In such a case, a physician will get access to several concentration estimates collected over a time period and can use this information for diagnostic purposes, such as detecting the onset of a medical condition or used in adjusting administration levels of medicaments.

The invention is based on the discovery that the frequency modulation of the transmitted signal depends on intrinsic body activity, such as breathing and cardiac beating. Such activity-dependency can be important for the interpretation of the frequency spectrum of the received RF signal. In order to reduce this signal blurring, the IMD 100 preferably synchronizes the act of transmitting a RF signal with variations in the intrinsic body activity.

The IMD 100 preferably has an electric signal analyzer 150 connected to at least one electrode-containing medical lead 210 through the connector 110. The signal analyzer 150 processes electric signals sensed by the electrode(s) 212, 214 of the lead 210 and originating from a body tissue, typically the heart. The analyzer 150 could, for instance, identify, based on the sensed electric signals, the start of a heart cycle, the systole phase of a heart cycle and the diastole phase of a heart cycle. In such a case, the processed electric signal can be regarded as an intracardiac electrogram (IEGM), from which such events can be detected by the signal analyzer 150.

An alternative example is an impedance signal generated by the signal analyzer 150. In such a case, the analyzer 150 has a signal applier arranged for applying a current or voltage signal to a particular tissue, such as heart, or over a portion of the patient body, using two electrodes 212, 214 positioned on one or more leads 210 connected to the IMD 100. The same electrodes 212, 214 or different electrodes can then be employed for sensing a resulting voltage or current signal. The analyzer 150 determines an impedance signal that registers variations in intrinsic body activity, such as heart cycle and/or respiration cycle, depending on the particular signal processing (filtering) of the sensed signal.

The RF signal controller 140 is then responsive to the representation of the variation in intrinsic body activity from the signal analyzer 150. This means that the controller 140 generates, based on the input from the analyzer 150, a synchronization signal that is forwarded to the signal source 120 and causes the source 120 to generate and forward a RF signal to the transmitting antenna 300. In this way, the signal transmission becomes synchronized to a particle phase of the intrinsic body activity, such as synchronized with inhalation, exhalation or systole, diastole. This means that for a periodic or regular analyte monitoring based on the RF spectrum analysis, the RF signal can be transmitted by the antenna 300 at corresponding phases of the intrinsic body activity but still at different time instances.

The frequency spectrum is not only depending on intrinsic body activities, such as heart beating and respiration, but also the body posture of the subject can affect the spectrum of the received RF signal. As a consequence, the IMD 100 can advantageously be equipped with or be connected to a body posture sensor 160. An example of such body posture sensors includes accelerometers, such as micro-electromechanical system (MEMS) based accelerometers. The sensor 160 can then generate a signal representative of the current body posture of the subject, e.g. by discriminating between standing and lying position or standing, sitting and lying position.

The signal controller 140 is then responsive to this body posture signal and generates based thereon a synchronization signal that is forwarded to the signal source 120. The source 120 generates a RF signal upon reception of the synchronization signal.

Having this body-posture synchronization capability, the signal controller 140 is able to limit RF signal transmissions to particular body postures to thereby obtain a consistency and body posture-independency in the RF signal processing, in particular for regular or periodic signal transmission and analysis.

The units 120 to 160 of the IMD 100 can be implemented in hardware, software of a combination of hardware and software.

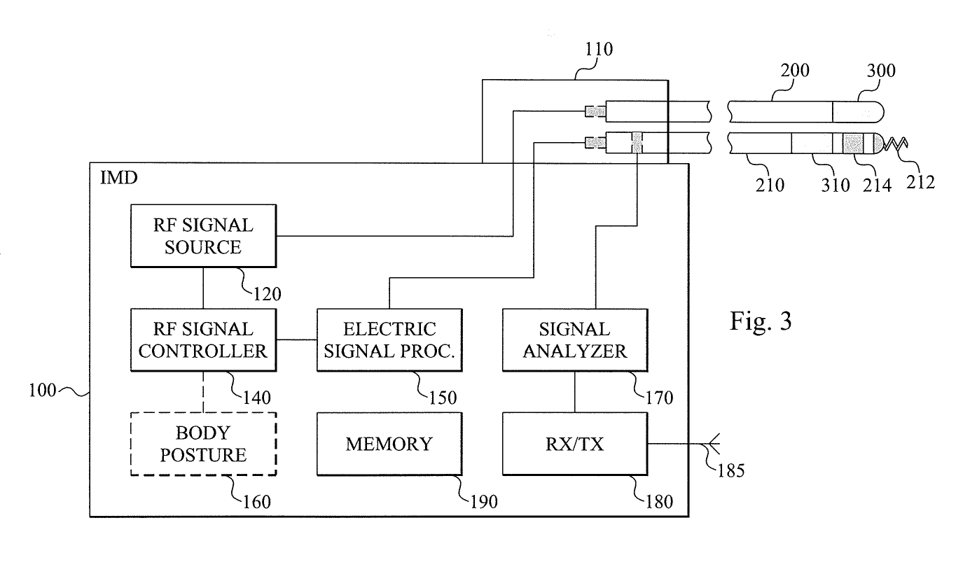

FIG. 3 illustrates another embodiment of the IMD 100 according to the present invention. In this embodiment, the IMD 100 does not include the signal processor that performs the processing of the received RF signal in order to generate the estimate or representation of the analyte concentration. The reason for this is that the spectral analysis performed by the processor can sometimes, depending on the particular analyte to monitor, be a quite complex task. In order to save power and thereby increase the operational time of the battery driven IMD 100, the processing can be performed by a non-implanted device that does not have the limited processing capability and limited power supply of the IMD 100. A typical example of such a non-implanted device is illustrated in FIG. 1 represented as the programmer.

In clear contrast, the IMD 100 instead has a signal analyzer 170 arranged for generating a representation of the received RF signal. This analyzer 170 can include traditional RF analyzing units, such as ND converter, encoder etc., for the purpose of generating a signal representation that can be further processed by a remote signal processor to get an estimate of the analyte concentration.

The signal analyzer 170 is connected to a transmitter 180 having a transmitting antenna 185 utilized for unidirectional or bidirectional communication with the non-implanted device. This antenna can actually be one of the transmitting 300 and receiving 310 antenna of the IMD 100 used for RF signal transmission and reception. Alternatively, the transmitter 180 has its dedicated antenna 185 that is only employed for communication with the external device and not for any diagnosing purposes. It is anticipated by the invention that this dedicated antenna 185 can be a RF antenna or an inductive antenna.

The units 120 to 150, 170 and 180 of the IMD 100 can be implemented in hardware, software of a combination of hardware and software.

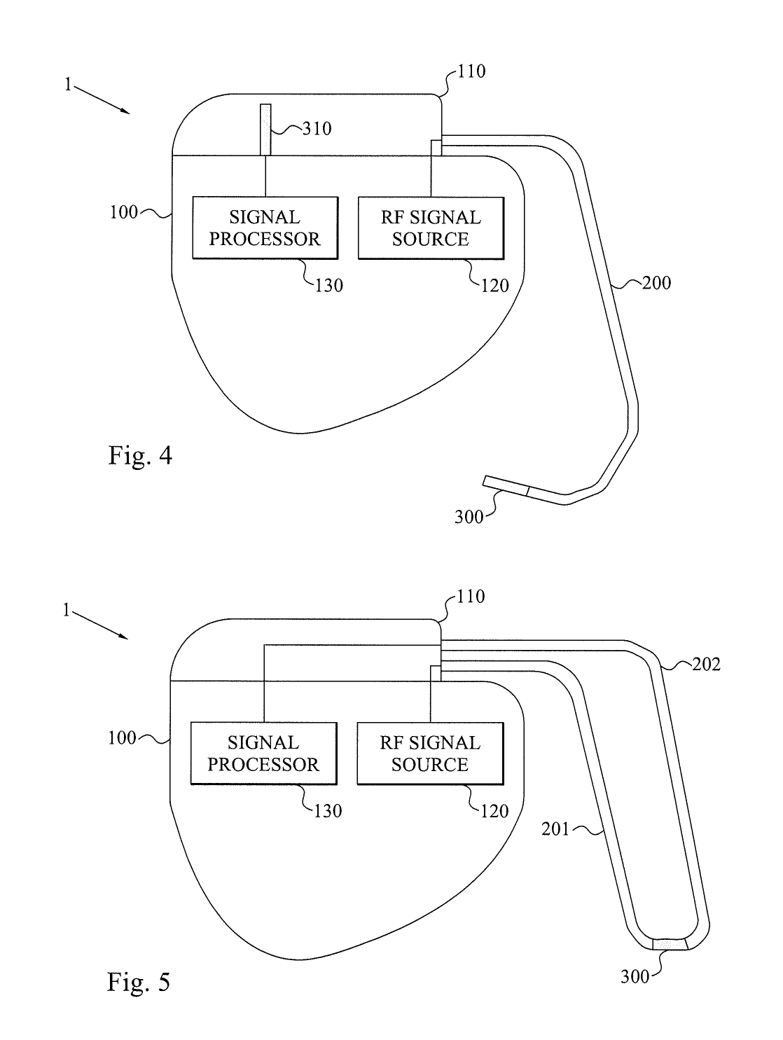

FIGS. 4-5, 7-9 illustrate different embodiments of analyte measuring systems 1 according to the present invention. As is evident from the disclosure below, the system 1 can include a single implantable medical device 100, multiple, i.e. at least two, implantable medical devices 100, 400 or an implantable medical device 100 and a non-implanted device 500. In these figures, the signal processor 130, 530 of the invention and the RF signal source 120, 420 have been indicated to illustrate the device in which they are implemented. However, respective device can include further units participating in the analyte measurement of the invention, such as the units illustrated in the IMD embodiments of FIGS. 2 and 3.

FIG. 4 illustrates the system 1 consisting of a single implantable medical device 100, such as a pacemaker, defibrillator or cardioverter. The IMD 100 therefore has both a transmitting antenna 300 and a receiving antenna 310. In this illustrative embodiment, the transmitting antenna 300 is provided on a transmission line 200 being connected to the IMD 100 through a connector 110. The transmission line 200 can be realized as a coaxial cable or some other antenna connecting catheter. The transmission line 200 is in connection with the RF signal source 120 arranged in the IMD 100.

FIG. 4 illustrates an alternative implementation for the receiving antenna 310. The antenna 310 is not arranged on any lead or transmission line but is instead an integral part of the IMD can 100. The antenna 310 is in connection, through the connector 110 to the signal processor 130 in the IMD 100.

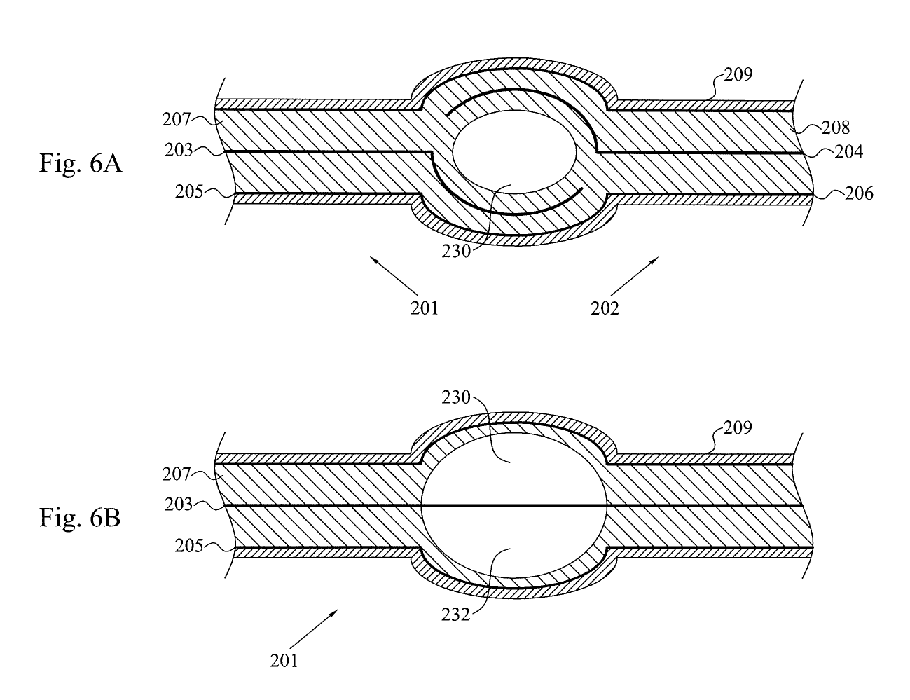

FIG. 5 also illustrates an IMD 100 based analyte measuring system 1 having the RF signal source 120 and the RF signal processor 130. FIG. 5 illustrates an alternative antenna embodiment that utilizes two transmission lines 201, 202 that could be two interconnected coaxial cables. In such a case, the signal measurements are performed in a transition region 310 between the two cables 201, 202. This transmission region may, as is illustrated in FIG. 6A, comprise of capacitively coupled extended center conductors 203, 204. A part of the fields that is providing the capacitive coupling is extending through the media or tissue to be measured, thus giving a measure of the absorption.

A first end of the first coaxial cable 201 is connected to the IMD 100 via the connector 110 and a first end of the second coaxial cable 202 is likewise connected to the IMD connector 110. The second opposite ends of the two cables 201, 202 are interconnected as illustrated in FIG. 6A. Thus, the outer conductors 205, 206 of the two cables 201, 202 are interconnected as is the dielectric 207, 208 between the inner 203, 204 and outer 205, 206 conductors and the outer insulating tubing 209. The ends of the inner conductors 203, 204 in this transition region are diametrically positioned on opposite sides of a diametric aperture 230 running transversely through the cables 201, 202 at their interconnection. If the analyte is to be measured in a blood volume, the transition region is preferably arranged in a blood vessel or in a heart chamber. As a consequence, blood can flow through the aperture 230 to form a local measurement.

FIG. 6B illustrates an alternative solution of providing a transmitting and receiving antenna utilizing a single coaxial cable 201. The cable 201 has its two ends connected to the connector arrangement 110 of the IMD 100 as illustrated in FIG. 5. In an intermediate cable portion, part of the dielectric 207 is removed forming two transverse channels 230, 232 between the inner conductor 203 and the outer conductor 205. As a consequence, blood may flow through these channels to obtain a local analyte measurement in the passing blood.

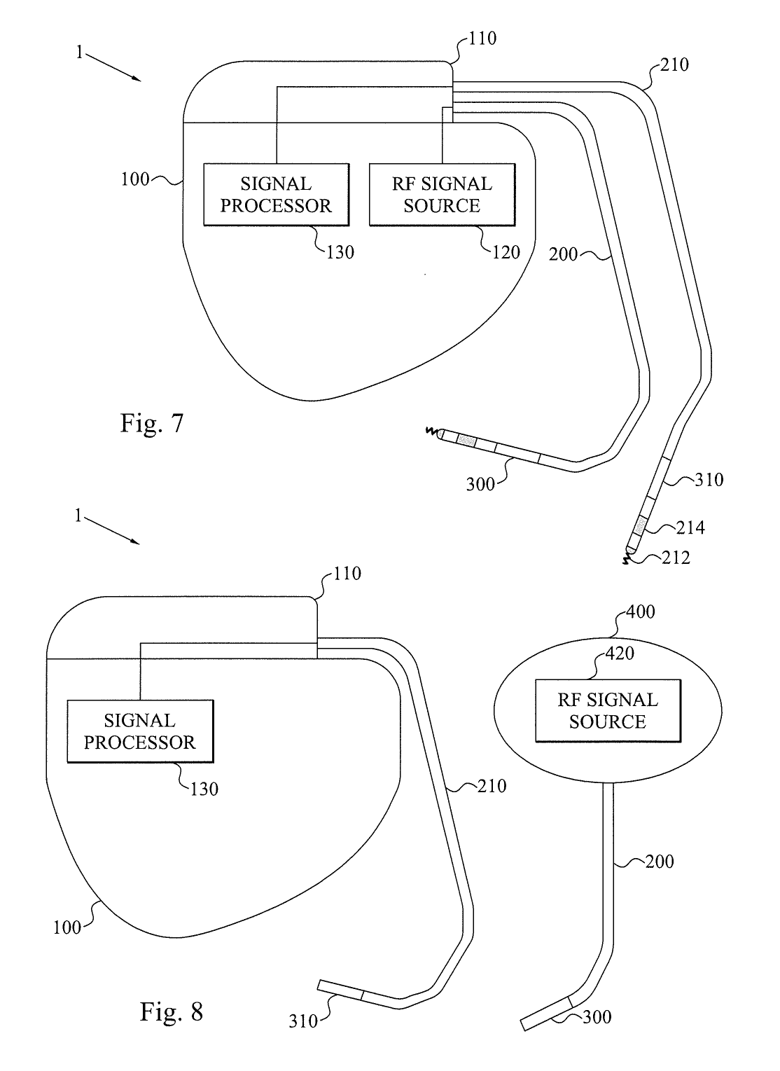

FIG. 7 illustrates the analyte measuring system 1 with an IMD 100 having two implantable medical leads 200, 210, each having at least one electrode 212, 214 employed for cardiac signal sensing and/or pulse or shock delivery. In this case, each lead 200, 210 also comprises a RF antenna 300, 310 according to the present invention. In such a case, the outer conducting wire or coil of the leads 200, 210 can be used as receiving 300 or transmitting 310 antenna element.

FIG. 8 illustrates a system embodiment 1 with two implantable medical devices 100, 400. A first transmitting device 100 can be a dedicated RF device basically including the RF signal source 420 and a transmission line 200 with transmitting antenna 300. The device 400 also has a power source, such as battery (not illustrated). An optional clock may also be included in the device 400 for usage by the signal source 420 in timing generation and transmission of the RF signal. The device 400 can be manufactured in small dimensions due to the limited number of functionalities that must be provided in the device 400.

The other IMD 100 includes the signal processor 130 or alternatively the signal analyzer 170 if the processor 130 is provided in a non-implanted device. The IMD 100 comprises a transmission line 210 having a receiving antenna 310 connected through the connector arrangement 110.

FIG. 9 illustrates a further embodiment of the analyte measuring system 1, in which one transmission part, either the transmitter as shown in FIG. 9 or the receiver, is implemented in the IMD 100. An external, non-implantable device 500, such as the programmer in FIG. 1, then houses the other transmission part. In the figure, the device 500 comprises the receiving antenna 310 connected to the signal processor 530. The transmitting antenna 300 and the RF signal source 120 are instead provided in or connected to the IMD 100.

In an alternative embodiment, the IMD 100 houses both the RF transmitting and receiving part but the spectral analysis is performed in the external device 500. As a consequence, device 500 has a receiver 520 with connected receiving antenna 525 for receiving a representation of the RF signal collected by the IMD 100. This RF signal representation is processed by the signal processor 530 of the device 500 for generating an analyte concentration estimate.

The present invention also encompasses different combinations of the embodiments illustrated in the previous figures as long as at least one of the transmission part, preferably the transmitter functionality, is implemented in an implantable medical device.

The present invention can be used in connection with different antenna implementations. However, the most preferable antenna from an antenna efficiency point of view should be sized a quarter of a wave length or larger in the tissue or media where it is immersed. The preferred frequency range, in which the RF signal bandwidth is selected, is 1 MHz to 1 THz. This corresponds to a wavelength of about 300 μm to 300 m. The most preferred range is from 100 MHz to 10 GHz, with the corresponding wavelengths of about 3 mm to about 3 m. A quarter wavelength is then from about 750 μm to 75 cm. Thus, it is actually possible, especially for the higher frequency range in the GHz area to, implant quarter wave RF antennas.

The antennas of the invention can be selected from, for instance, the following antenna types: a leaky wave antenna, a slotted waveguide antenna, an electrical dipole antenna, a magnetic dipole antenna, a patch antenna, a planar inverted-F antenna (PIFA), an inverted-F antenna (IFA), and an inverted-L antenna (ILA).

The analyte measured and monitored according to the present invention is an analyte present in a tissue of a subject body. In a preferred embodiment, the tissue is blood so that the analyte concentration is then the blood concentration. Other preferred tissues include cardiogenic tissue, such as myocardium. As a consequence, the RF transmitting and receiving antennas of the present invention are preferably placed in or in connection with the tissue in which the analyte is to be monitored. However, the advantage of using RF signals of the present invention is that the antennas do not necessarily have to be place directly or in immediate connection with the measuring tissue. As long as the RF signal are able to penetrate through the tissue and be captured by the receiving antenna, thereby achieving a more unrestricted placement of the antennas. The present invention therefore has the further advantage that the implantation site of the antennas can be selected mainly from surgical and anatomical points of view instead of mainly being limited to the site of the tissue of interest.

The RF signal measurements of the present invention can be used both for local analyte measurements, such as when utilizing the antenna embodiments illustrated in FIGS. 6A and 6B, and for more global or regional analyte measurements. In the latter case, the receiving and transmitting antennas are arranged in spatially different sited in the subject body or one of the antennas are not implanted.

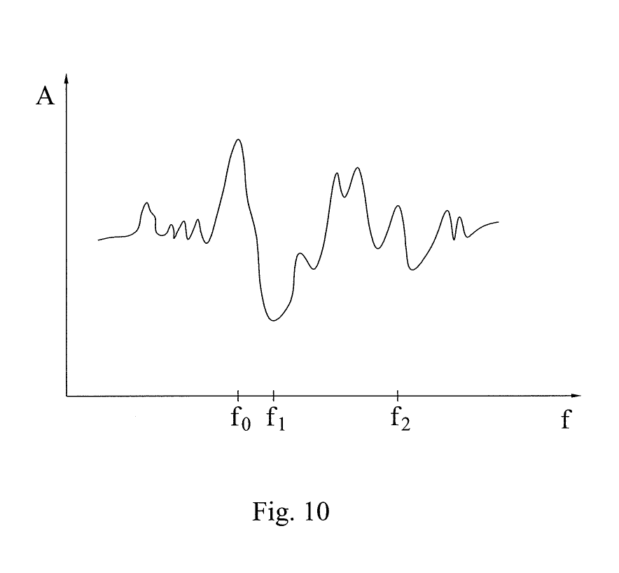

FIG. 10 is an example of an absorbance spectrum that could be obtained by the analyte measuring system of the present invention. As can be seen from FIG. 10, such an absorbance spectrum will exhibit peaks of a global and local maximums and valleys of a global and local minimums depending on what analytes that are present in a test tissue and in what concentrations the analytes are present. The interesting frequencies (f₀, f₁) that are relevant for a given analyte and that should be investigated in the spectrum analysis of the invention can be defined in advance based on laboratory tests and/or mathematical estimations of absorbance/resonance frequency based on the three-dimensional structure of the analyte and its constituents.

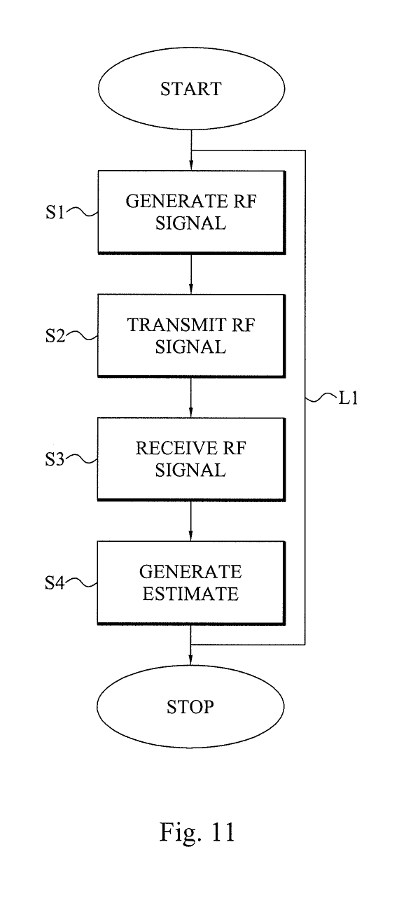

FIG. 11 is a flow diagram of a method for estimating an analyte according to the present invention. The method starts in step S1, where a RF signal of selected bandwidth is generated by a RF signal source, preferably arranged in an IMD. A next step S2 transmits the RF signal by a transmitting antenna, preferably an implantable transmitting antenna into a surrounding tissue or medium in an animal, preferably mammalian and more preferably human, body. A (implantable or non-implantable) receiving antenna receives the resulting RF signal from the surrounding tissue in step S3. Finally, step S4 generates an estimate or representation of the concentration of the analyte in the surrounding tissue based on a spectral analysis of the RF signal received by the receiving RF antenna.

The analyte measurements of steps S1 to S4 are preferably performed at multiple different time instances, such as intermittently or periodically, which is schematically illustrated by the line L1. The time interval between measurements is mainly dependent on the particular analyte to monitor and can non-inventively be selected by a physician. For some analytes, a periodic measurement once every hour or even more often could be advantageous at least during a limited time interval. For other analytes, one or a few measurements per day, week or month could be adequate.

The present invention is not limited to measuring and monitoring a single analyte. In clear contrast, by varying the frequency of the transmitted RF signal to adapt to absorption and/or resonance frequencies of different analytes, it is actually possible to measure and monitor concentrations and concentration changes of multiple different analytes in a subject body. Furthermore, the periodicity in such measurements can be different for the different analytes.

Although modifications and changes may be suggested by those skilled in the art, it is the intention of the inventors to embody within the patent warranted heron all changes and modifications as reasonably and properly come within the scope of their contribution to the art.

We claim as our invention:

1. An analyte measuring system comprising:
an implantable medical device comprising:
a radio frequency, RF, signal source that generates at least one RF signal of a RF range;
a transmitting RF antenna connected to said frequency tunable source that transmits said at least one RF signal into surrounding tissue;
a receiving RF antenna that receives said at least one RF signal from said surrounding tissue; and
a signal processor in or in communication with the implantable device that generates an estimate of a concentration of said analyte in said surrounding tissue by making a spectral analysis of said at least one RF signal received by said receiving antenna.

2. The system according to claim 1, wherein said RF signal source is a frequency tunable RF signal source that sweeps a frequency of said at least one RF signal to obtain multiple RF signals of different frequencies in said RF range and said signal processor generates said estimate of said concentration of said analyte based on an analysis of at least a portion of said multiple RF signals.

3. The system according to claim 1, wherein said RF signal source generates a composite RF signal having a defined bandwidth covering said RF range, and said signal processor generates said estimate of said concentration of said analyte based on an analysis of said RF signal at multiple selected frequencies in said RF range obtained by bandpass filtering said composite RF signal.

4. The system according to claim 1, wherein said transmitting RF antenna is arranged on an implantable electrical lead connected to said implantable medical device.

5. The system according to claim 1, wherein said receiving RF antenna is connected to said implantable medical device.

6. The system according to claim 5, wherein said receiving RF antenna is arranged on an implantable electrical lead connected to said implantable medical device.

7. The system according to claim 5, wherein said implantable medical device comprises:
a signal analyzer that generates a representation of said at least one RF signal received by said receiving antenna; and
a second transmitting antenna that transmits said representation to a non-implantable device comprising a second receiving antenna that receives said representation, said signal processor being arranged in said non-implantable device.

8. The system according to claim 5, wherein said transmitting RF antenna and said receiving RF antenna are collectively formed by a first coaxial cable having a first end connected to said implantable medical device and a second end connected to a first end of a second coaxial cable having a second end connected to said implantable medical device, a first center conductor of said first coaxial cable being capacitively coupled to a second center conductor of said second coaxial cable.

9. The system according to claim 8, wherein a first end of said first center conductor is connected to said implantable medical device and a second end of said end of said first center conductor is radially provided on a first side of a diametric aperture formed in the interconnection of said first and second coaxial cables and a first end of said second center conductor is connected to said implantable medical device and a second end of said second center conductor is radially provided on a second opposite side of said diametric aperture.

10. The system according to claim 5, wherein said transmitting RF antenna and said receiving RF antenna are collectively formed by a coaxial cable having a first end connected to said implantable medical device and a second end connected to said implantable medical device, an intermediate portion of said coaxial cable exhibiting two transverse channels provided between an outer conductor of said coaxial cable and a center conductor of said coaxial cable.

11. The system according to claim 1, wherein said transmitting RF antenna and said receiving RF antenna are selected from the group consisting of:
a leaky wave antenna;
a slotted waveguide antenna;
an electrical dipole antenna;
a magnetic dipole antenna;
a patch antenna;
a planar inverted-F antenna, PIFA;
an inverted-F antenna, IFA; and
an inverted-L antenna, ILA.

12. The system according to claim 1, wherein said signal processor is arranged in said implantable medical device connected to said receiving RF antenna.

13. The system according to claim 1, wherein said signal processor generates an estimate of a concentration of a peptide or protein analyte in said surrounding tissue based on said spectral analysis.

14. The system according to claim 13, wherein said peptide or protein analyte is selected from the group consisting of:
B-type natriuretic peptide, BNP;
atrial natriuretic peptide, ANP;
troponin;
myoglobin; and
creatine kinase.

15. The system according to claim 1, wherein said RF signal source generates said at least one RF signal of a RF range comprising an absorbance frequency characteristic of said analyte.

16. The system according to claim 1, wherein said RF signal source generates said at least one RF signal of said RF range in a frequency window of 1 MHz to 1 THz.

17. The system according to claim 16, wherein said RF signal source generates said at least one RF signal of said RF range in a frequency window of 10 MHz to 10 GHz.

18. The system according to claim 17, wherein said RF signal source generates said at least one RF signal of said RF range in a frequency window of 100 MHz to 10 GHz.

19. The system according to claim 1, wherein said signal processor generates said estimate of said analyte concentration based on a ratio an amplitude of said at least received RF signal at a first frequency and an amplitude of said at least one received RF signal at a second frequency.

20. The system according to claim 19, wherein said signal processor generates said estimate of said analyte concentration based on a comparison of said ratio and a corresponding ratio determined by said signal processor based on a spectrum analysis of a RF signal previously received by said receiving antenna.

21. The system according to claim 1, wherein said implantable medical device further comprises:
at least one implantable electrical lead having at least one electrode for sensing an electrical signal from a surrounding tissue;
a signal analyzer that generates a representation of a variation in intrinsic body activity based on said sensed electrical signal; and
a signal control unit that generates a synchronization signal based on said representation of said variation in intrinsic body activity and forwards said synchronization signal to said RF signal source, said RF signal source responding to said synchronization signal by generating said at least one RF signal based on said synchronization signal.

22. The system according to claim 1, wherein said implantable medical device further comprises:
a body posture sensor that generates a representation of a body posture of a subject in whom said implantable medical device is implanted; and
a signal control unit that generates a synchronization signal based on said representation of said body posture and forwards said synchronization signal to said RF signal source, said RF signal source responding to said synchronization signal that generates said at least one RF signal based on said synchronization signal.

23. The system according to claim 1, wherein said implantable medical device is selected from the group consisting of:
a pacemaker;
a cardiac defibrillator; and
a cardioverter.

24. An analyte estimating method comprising:
generating at least one radio frequency, RF, signal of a RF range;
transmitting said at least one RF signal by an implantable transmitting RF antenna into a surrounding tissue;
receiving said at least one RF signal from said surrounding tissue by a receiving RF antenna; and
generating an estimate of a concentration of said analyte in said surrounding tissue by making a spectral analysis of said at least one RF signal received by said receiving RF antenna (310).

25. The method according to claim 24, wherein said signal generating step comprises sweeping a frequency of said at least one RF signal to obtain multiple RF signals of different frequencies in said RF range and said estimate generating step comprises generating said estimate of said concentration of said analyte based on an analysis of at least a portion of said multiple RF signals.

26. The method according to claim 24, wherein said signal generating step comprises generating a composite RF signal having a defined bandwidth covering said RF range and said estimate generating step comprises generating said estimate of said concentration of said analyte based on an analysis of said RF signal at multiple selected frequencies in said RF range obtained by bandpass filtering said composite RF signal.
